(19) United States
(12) Patent Application Publication
(10) Pub. No.: US 2024/0201152 A1
(43) Pub. Date: Jun. 20, 2024

(54) GAS DETECTOR DEVICE ACCESSORY IDENTIFICATION AND ANALYSIS

(71) Applicant: MSA TECHNOLOGY, LLC, Cranberry Township, PA (US)

(72) Inventors: Daniel Miller, Cranberry Township, PA (US); Shane Kelleher, Glenshaw, PA (US); Minh Pham, Slippery Rock, PA (US); Anthony Bafile, Jeannette, PA (US)

(21) Appl. No.: 18/083,484

(22) Filed: Dec. 17, 2022

(51) Int. Cl. G01N 33/00; G01N 1/24; G06K 7/10; G06K 19/07

(52) U.S. Cl. CPC G01N 33/0027; G01N 1/24; G06K 7/10366; G06K 19/0723

(57) ABSTRACT

A system includes a gas detection device and an accessory device identification system in communicative connection with a control system of the gas detection device. The accessory device identification system is configured to receive data including identification data for at least one of (i) each of one or more associated accessory devices or (ii) an associated combination of accessory devices configured to be placed in fluid connection with the inlet of the gas detection device. The accessory device identification system is configured to identify at least one of (i) each of the one or more associated accessory devices or (ii) the associated combination of accessory devices (from the identification data received.

### BACKGROUND

The following information is provided to assist the reader in understanding technologies disclosed below and the environment in which such technologies may typically be used. The terms used herein are not intended to be limited to any particular narrow interpretation unless clearly stated otherwise in this document. References set forth herein may facilitate understanding of the technologies or the background thereof. The disclosure of all references cited herein are incorporated by reference.

Gas detection/detector devices (often referred to as “instruments”) include at least one gas sensor, electronic circuitry, and a power supply to drive the sensor, interpret its response, and display its response to the user. Such gas detection devices or instruments may include a variety of sensors for detecting gas analytes including, for example, electrochemical gas sensors and combustible gas sensors. See, for example, U.S. Pat. No. 9,784,755, the disclosure of which is incorporated herein by reference. Gas detection devices further include a housing to enclose and protect such components. Gas detection devices may be portable or fixed in position. Portable gas detection devices are typically powered by a rechargeable battery system. Portable gas detection devices are carried on the person of an authorized user or worker within a company/facility that may own or rent the portable gas detection devices as an asset of the facility. Depending upon size and industry, a company/facility may, for example, have several, tens, or hundreds or more of such devices that are shared amongst a large, dynamic, and transitory workforce.

Accessories available in the gas detection market for use with, for example, gas detection devices, are numerous and place significant responsibility on the end user to ensure proper use. Accessories, including pump probes, sampling lines of various lengths, materials, and use cases, as well as adapters, are typically used in pumped instrument applications where the instrument is being used as a tool for designation of gas levels in a specific area, and in many cases a specific gas or volatile organic compound (VOC).

Under current practice, the responsibility to select the correct accessory device or accessory device combinations (sometimes referred to as a stack) which form an accessory device transport path or accessory device assembly for the sensor configuration of the gas detection device or instrument falls on the user. To complicate this issue, the market is flooded with accessory devices such as sampling lines, probes and adapters from multiple device manufacturers from present and prior generations of products as such accessory devices are rarely obsoleted or pushed out of service.

### SUMMARY

In one aspect, a system includes a gas detection device including a housing, a control system including a processor system and a memory system in communicative connection with the processor system, and one or more gas sensors within the housing in communicative connection with the control system. Each of the one or more gas sensors is independently responsive to a gas analyte. The gas detection device further includes an inlet via which gas to be sampled from an environment enters the housing to come into contact with the one or more gas sensors and a pump system in fluid connection with the inlet and in fluid connection with the one or more gas sensors. The system further includes an accessory device identification system in communicative connection with the control system of the gas detection device. The accessory device identification system is configured to receive data including identification data for an associated accessory device assembly including one or more gas accessory devices. In that regard, the accessory device identification system is configured to receive data including identification data for at least one of (i) each of one or more associated accessory devices or (ii) an associated combination of accessory devices configured to be placed in fluid connection with the inlet of the gas detection device. The accessory device identification system is configured to identify at least one of (i) each of the one or more associated accessory devices or (ii) the associated combination of accessory devices (that is, to identify the associated accessory device assembly) from the identification data received.

The accessory device identification system may further include a database having stored therein data for at least one of a plurality of accessory devices or a plurality of combinations of accessory devices. The stored data includes reference identification data for at least one of each of the plurality of accessory devices or each of the plurality of combinations of accessory devices. The accessory device identification system may further be configured to compare the identification data received with the reference identification data to identify (i) each of the one or more associated accessory devices or (ii) the associated combination of accessory devices.

The database may further have stored therein characterization data for at least one of each of the plurality of accessory devices or each of the plurality of combinations of accessory devices. In a number of embodiments, the database is stored in the memory system of the control system. One or more of (or each of) the one or more associated accessory devices or the associated combination of accessory devices may, for example, include an identifier in operative connection therewith. The identifier may, for example, be configured to provide the data including the identification data to the accessory device identification system. In a number of embodiments, the identifier is configured to provide the data including the identification data to the accessory device identification system via a communication device of the accessory device identification system. In a number of embodiments, the identifier includes an RFID tag and the communication device of the accessory device identification system includes an RFID reader.

In a number of embodiments, the gas detection device further includes a sensing system in operative connection with the control system. The sensing system is configured to measure a property of the gas detection device (for example, a property, operational parameter, or operational variable of the pump system thereof) which varies upon attachment of the one or more associated accessory devices or the associated combination of accessory devices to the inlet. Data of the property of the pump system of the gas detection device may, for example, be used in at least one of: identifying the one or more associated accessory devices or the associated combination of accessory devices, wherein the reference identification data comprises the data of the property of the pump system, determining a state of attachment of the one or more associated accessory devices or the associated combination of accessory devices, or determining a state of functionality of the one or more associated accessory devices or the associated combination of accessory devices.

The memory system may further include software stored thereon and executable by the processor system to compare the identification data received with the reference identification data to identify the one or more associated accessory devices or the associated combination of accessory devices. In a number of embodiments, the database further has stored therein characterization data for at least one of the plurality of accessory devices or the plurality of combinations of accessory devices. The memory system may further include analysis software stored thereon which is executable by the processor system to analyze the one or more associated accessory devices or the associated combination of accessory devices. In a number of embodiments, the analysis software is configured to determine at least one of compatibility of the one or more associated accessory devices or the associated combination of accessory devices with the gas detection device, the compatibility of one associated accessory devices with another associated accessory devices, compatibility of the one or more associated accessory devices or the associated combination of accessory devices with a location, compatibility of the one or more associated accessory devices or the associated combination of accessory devices with a user, compatibility of the one or more associated accessory devices or the associated combination of accessory devices with an environment to be tested, a state of connection of the one or more associated accessory devices or the associated combination of accessory devices to the inlet, a parameter of operation of the gas detection device for use with the one or more associated accessory devices or the associated combination of accessory devices, a recommendation for changing at least one associated accessory device, or a change in operation of the portable gas detection device.

In a number of embodiments, the system further includes a management system including a management processor system, a management communication system configured to be in communicative connection with a gas monitoring system of an entity which includes the gas detection device, and a management memory system in operative connection with the management processor system. The management memory system further includes or has stored therein an algorithm executable by the management processor system to at least one of track usage of or manage the gas detection device and at least one of the one or more associated accessory devices or the associated combination of accessory devices.

In another aspect, a method for use with a gas detection device includes providing an accessory device identification system in communicative connection with a control system of the gas detection device, the accessory device identification system being configured to receive data including identification data for at least one of (i) each of one or more associated accessory devices or (ii) an associated combination of accessory devices configured to be placed in fluid connection with the inlet of the gas detection device, and identifying at least one of (i) each of the one or more associated accessory devices or (ii) the associated combination of accessory devices from the identification data received. The gas detection device includes a housing, the control system, which may include a processor system and a memory system in communicative connection with the processor system, and one or more gas sensors within the housing in communicative connection with the control system. Each of the one or more gas sensors is independently responsive to a gas analyte. Th gas detection device further include an inlet via which gas to be sampled from an environment enters the housing to come into contact with the one or more gas sensors and a pump system in fluid connection with the inlet and in fluid connection with the one or more gas sensors.

In a number of embodiments, the accessory device identification system includes a database having stored therein data for at least one of a plurality of accessory devices or a plurality of combinations of accessory devices including reference identification data for at least one of each of the plurality of accessory devices or each of the plurality of combinations of accessory devices. The accessory device identification system may further be configured to compare the identification data received with the reference identification data to identify (i) each of the one or more associated accessory devices or (ii) the associated combination of accessory devices. The database may further have stored therein characterization data for at least one of each of the plurality of accessory devices or each of the plurality of combinations of accessory devices. The database may be stored in the memory system of the control system.

In a number of embodiments, one or more of (or each of) the one or more associated accessory devices or the associated combination of accessory devices may, for example, include an identifier in operative connection therewith. The identifier may, for example, be configured to provide the data including the identification data to the accessory device identification system. In a number of embodiments, the identifier is configured to provide the data including the identification data to the accessory device identification system via a communication device of the accessory device identification system. In a number of embodiments, the identifier includes an RFID tag and the communication device of the accessory device identification system includes an RFID reader.

The gas detection device may further include a sensing system in operative connection with the control system and configured to measure a property of the gas detection device (for example, a property of the pump system thereof) which varies upon attachment of the one or more associated accessory devices or the associated combination of accessory devices to the inlet. The method may further include using the data of the property of the pump system to at least one of: (i) identify the one or more associated accessory devices or the associated combination of accessory devices, (ii) determine a state of attachment thereof, or (iii) determine a state of functionality thereof.

The memory system may further have software stored thereon and executable by the processor system to compare the identification data received with the reference identification data to identify the one or more associated accessory devices or the associated combination of accessory devices. In a number of embodiments, the database further has stored therein characterization data for at least one of the plurality of accessory devices or the plurality of combinations of accessory devices. The method may further include analyzing the one or more associated accessory devices or the associated combination of accessory devices via analysis software stored in the memory system which is executable by the processor system to analyze the one or more associated accessory devices or the associated combination of accessory devices. The analysis software may, for example, be configured to determine at least one of compatibility of the one or more associated accessory devices or the associated combination of accessory devices with the gas detection device, the compatibility of one associated accessory devices with another associated accessory devices, compatibility of the one or more associated accessory devices or the associated combination of accessory devices with a location, compatibility of the one or more associated accessory devices or the associated combination of accessory devices with a user, compatibility of the one or more associated accessory devices or the associated combination of accessory devices with an environment to be tested, a state of connection of the one or more associated accessory devices or the associated combination of accessory devices to the inlet, a parameter of operation of the gas detection device for use with the one or more associated accessory devices or the associated combination of accessory devices, a recommendation for changing at least one associated accessory device, or a change in operation of the portable gas detection device.

The method may further include at least one of tracking usage of or managing the gas detection device and at least one of the one or more associated accessory devices or the associated combination of accessory devices via a software-based management system.

The devices, systems, and methods hereof, along with the attributes and attendant advantages thereof, will best be appreciated and understood in view of the following detailed description taken in conjunction with the accompanying drawings.

### BRIEF DESCRIPTION OF THE DRAWINGS

FIG. 1A illustrates a front isometric view of a representative embodiment of a gas detection instrument, device or detector for use herein.

FIG. 1B illustrates an isometric, exploded or disassembled view of the gas detection instrument of FIG. 1A.

FIG. 2 illustrates schematically communication in an embodiment of an instrument management grid or network hereof.

FIG. 3A illustrates an embodiment of a sampling line accessory for use with, for example, the gas detection instrument of FIG. 1A.

FIG. 3B illustrates an embodiment of a sampling line and a connected probe accessory or use with, for example, the gas detection instrument of FIG. 1A.

FIG. 3C illustrates schematically different line lengths and diameters of sampling lines for use in connection with, for example, the gas detection instrument of FIG. 1A.

FIG. 4A illustrates a system including the gas detection instrument of FIG. 1A, a sampling line, and a probe.

FIG. 4B illustrates use a representative use of the system of FIG. 4A in a facility.

FIG. 5 illustrates a workflow diagram of actions carried out in preparation detection of gas in a confined space to the point of travelling to the work site.

FIG. 6A illustrates a workflow diagram of actions carried out under typical current practices after a user arrives at a site and the user attaches accessories to a gas detection instrument.

FIG. 6B illustrates an embodiment of a workflow diagram after a user arrives at a site using devices, system, and methods for accessory device identification and analysis in connection with attachment of accessory device(s) to a gas detection device which is an asset in a networked grid.

### DETAILED DESCRIPTION

It will be readily understood that the components of the embodiments, as generally described and illustrated in the figures herein, may be arranged and designed in a wide variety of different configurations in addition to the described representative embodiments. Thus, the following more detailed description of the representative embodiments, as illustrated in the figures, is not intended to limit the scope of the embodiments, as claimed, but is merely illustrative of representative embodiments.

Reference throughout this specification to “one embodiment” or “an embodiment” (or the like) means that a particular feature, structure, or characteristic described in connection with the embodiment is included in at least one embodiment. Thus, the appearance of the phrases “in one embodiment” or “in an embodiment” or the like in various places throughout this specification are not necessarily all referring to the same embodiment.

Furthermore, described features, structures, or characteristics may be combined in any suitable manner in one or more embodiments. In the following description, numerous specific details are provided to give a thorough understanding of embodiments. One skilled in the relevant art will recognize, however, that the various embodiments can be practiced without one or more of the specific details, or with other methods, components, materials, et cetera. In other instances, well known structures, materials, or operations are not shown or described in detail to avoid obfuscation.

As used herein and in the appended claims, the singular forms “a,” “an”, and “the” include plural references unless the context clearly dictates otherwise. Thus, for example, reference to “an accessory device” includes a plurality of such accessory devices and equivalents thereof known to those skilled in the art, and so forth, and reference to “the accessory device” is a reference to one or more such accessory devices and equivalents thereof known to those skilled in the art, and so forth. Recitation of ranges of values herein are merely intended to serve as a shorthand method of referring individually to each separate value falling within the range. Unless otherwise indicated herein, and each separate value, as well as intermediate ranges, are incorporated into the specification as if individually recited herein. All methods described herein can be performed in any suitable order unless otherwise indicated herein or otherwise clearly contraindicated by the text.

The terms “electronic circuitry”, “circuitry” or “circuit,” as used herein include, but is not limited to, hardware, firmware, software, or combinations of each to perform a function(s) or an action(s). For example, based on a desired feature or need. a circuit may include a software-controlled microprocessor, discrete logic such as an application specific integrated circuit (ASIC), or other programmed logic device. A circuit may also be fully embodied as software. As used herein, “circuit” is considered synonymous with “logic.” The term “logic”, as used herein includes, but is not limited to, hardware, firmware, software, or combinations of each to perform a function(s) or an action(s), or to cause a function or action from another component. For example, based on a desired application or need, logic may include a software-controlled microprocessor, discrete logic such as an application specific integrated circuit (ASIC), or other programmed logic device. Logic may also be fully embodied as software.

The term “processor,” as used herein includes, but is not limited to, one or more of virtually any number of processor systems or stand-alone processors, such as microprocessors, microcontrollers, central processing units (CPUs), and digital signal processors (DSPs), in any combination. The processor may be associated with various other circuits that support operation of the processor, such as random-access memory (RAM), read-only memory (ROM), programmable read-only memory (PROM), erasable programmable read only memory (EPROM), clocks, decoders, memory controllers, or interrupt controllers, etc. These support circuits may be internal or external to the processor or its associated electronic packaging. The support circuits are in operative communication with the processor. The support circuits are not necessarily shown separate from the processor in block diagrams or other drawings.

The term “controller,” as used herein includes, but is not limited to, any circuit or device that coordinates and controls the operation of one or more input and/or output devices. A controller may, for example, include a device having one or more processors, microprocessors, or central processing units capable of being programmed to perform functions.

The term “software,” as used herein includes, but is not limited to, one or more computer readable or executable instructions that cause a computer or other electronic device to perform functions, actions, or behave in a desired manner. The instructions may be embodied in various forms such as routines, algorithms, modules, or programs including separate applications or code from dynamically linked libraries. Software may also be implemented in various forms such as a stand-alone program, a function call, a servlet, an applet, instructions stored in a memory, part of an operating system or other type of executable instructions. It will be appreciated by one of ordinary skill in the art that the form of software is dependent on, for example, requirements of a desired application, the environment it runs on, or the desires of a designer/programmer or the like.

As used herein, the term “personal communications device” refers to a portable or mobile device which includes a communication system, a processor system, a user interface system (for example, a visual feedback system including a touchscreen or other display, an auditory feedback system, and a tactile feedback system, a user input system etc.) and an operating system capable of running general-purpose applications. Examples of personal communications devices include, but are not limited to, smartphones, tablet computer and custom devices. As used herein, the term “tablet computer” or tablet, refers to a mobile computer with a communication system, a processor system, at least one user interface as described above (typically including a touchscreen display), and an operating system capable of running general-purpose applications in a single unit. As used herein, the term “smartphone” refers to a cellular telephone including a processor system, at least one user interface as described above (typically including a touchscreen display), and an operating system capable of running general-purpose applications. Such personal communication devices are typically powered by rechargeable batteries and are housed as a single, mobile unit. Moreover, in a number of embodiments personal communications devices are able accept input directly into a touchscreen (as opposed to requiring a keyboard and/or a mouse). Personal communications devices as typically provide for internet access through cellular networks and/or wireless internet access points connected to routers. A number of representative embodiments of systems and/or methods hereof are discussed in connection with the user of a smartphone as the personal communication instrument or device is described as a component of a system which includes.

As used herein, the term “database” refers an organized collection of structured information, or data, typically stored electronically in a memory system of a computer system.

In a number of embodiments, the devices, systems, and methods hereof are used to identify one or more associated accessory devices which are configured to be placed in fluid connection with or assembled upon the inlet of the portable gas detection device. One or more accessory devices are sometimes referred to herein as an accessory device assembly or an assembly. The one or more accessory devices may be associated with a portable gas detection device hereof by, for example, be placed in proximity to the gas detection device hereof or in operative connection with the gas detection device. The devices, systems, and method hereof may be at least partially or fully incorporated/integrated into a portable gas detection device hereof. One or more functions or components of the devices, systems, and method hereof may additionally or alternatively be incorporated or integrated into one or more components used in connection with a portable gas detection device (for example, in a networked system or grid). In a number of embodiments, each of the one or more accessory devices may include an identifier configured to transmit provide data including identification data for the accessory device to the accessory device identification system to be identified individually. In other embodiments, a combination or stack of accessory items may, for example, be provided as a kit and a single identifier may be associated with the combination to transmit or provide data including identification data for the combination or stack. Alternatively or additionally, the accessory device identification system may include a sensing system (for example, a sensor or electronic circuitry) configured to measure an operational variable or property of gas detection device (for example, an operational variable associated with the pump system thereof) which varies upon attachment of at least one of the one or more accessory devices to the inlet. The value of such a property may be used by the accessory device identification system to identify the accessory device(s) or combinations thereof, to confirm identification, to test or confirm operational/proper attachment, and/or to test or confirm functionality of the accessory device(s). Upon identification of the one or more accessory devices, characterization data stored in a memory system of the accessory device identification system for the identified accessory device(s) may be used for analysis. As used herein, “characterization data” includes any data associated with an identified accessory device assembly (which includes one or more accessory devices) that can be used in analysis of accessory device assembly. Such analysis may, for example, include checking for compatibility with the gas detection device, checking for compatibility with a location, checking for compatibility with a user, checking for compatibility with one or more gases in an environment, checking for proper installation, recommending alteration of accessory devices and/or operation of the portable gas detection devices, altering operation of the portable gas detection device, etc.

In representative embodiments discussed herein, a portable gas detection device is described as being in communicative connection with a network or grid of devices or systems to, for example, provide software-based assistance in managing the gas detection instrument or device or a plurality/fleet thereof. As described above, the accessory device identification and analysis functionality hereof may be fully integrated within the gas detection device or distributed over various components of such a network or another type of network. Furthermore, the accessory device identification and analysis functionality hereof may provide for and facilitate tracking and management of various gas detection accessory devices of a facility via software-based management system in operative or communicative connection with the gas detection instrument or device.

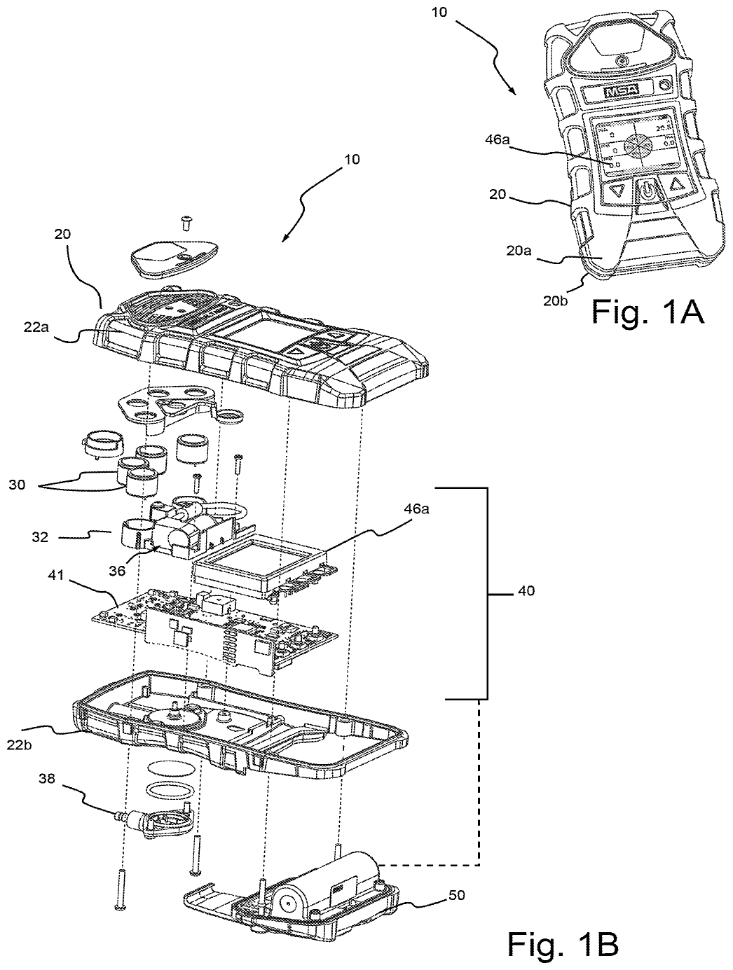

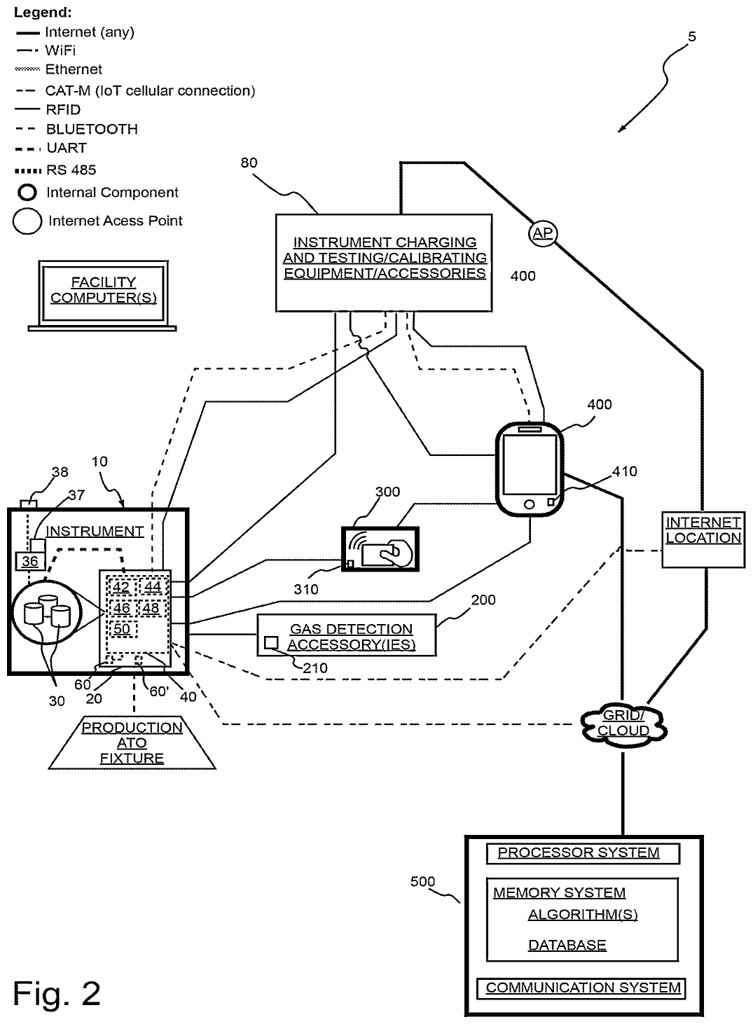

FIGS. 1A, 1B and 2 illustrates an embodiment of a portable instrument, detector device, or device 10 including a housing 20 formed from housing sections 22a and 22b to encompass one or more sensors 30 which may be seated within a sensor bracket 32. Bracket 32 also supports a pump system 36 in fluid connection with inlet 38. Each sensor is operable to detect the presence of an analyte. Sensors 30 are placed in connection with an environment to be tested via inlet 38.

Device 10 further includes electronic circuitry 40 including a controller or control system to control operation of device or instrument 10 and to analyze or interpret the responses of the sensor(s) 30. As illustrated schematically in FIG. 2, the control system may, for example, include a processor system 42 (for example, including one or more microprocessors) in operative connection with a memory system 44. As illustrated in FIG. 1B, elements of electronic circuitry can be incorporated in one or more printed circuit boards 41. One or more software algorithms may be stored in memory system 44 which is/are executable by processor system 42 to control/operate device 10 including, for example, data measurement/acquisition, analysis, device accessory identification and analysis as described herein, communication etc. A user interface system 46 (including, for example, a display 46a (see FIG. 1B), a speaker, a tactile system, various input/output systems, etc.) may also be placed in operative or communicative connection with processor system 42. A communication system 48 is in operative or communicative connection with processor system 42 for wired and/or wireless communication to other devices/systems. A power source 50 (for example, a battery system including one or more lithium batteries) provides power for electronic circuitry 40. Gas sensor(s) 30 may, for example, be placed in operative or communicative connection with electronic circuitry 40 via Universal Asynchronous Receive/Transmitter or UART protocol which may, for example, be a part of integrated circuit (IC) used for serial communications over a serial port.

As, for example, described in U.S. patent application Ser. No. 17/496,420, the disclosure of which is incorporated herein by reference, gas detection instrument or device 10 may, in a number of embodiments, be in communicative connection with a network or grid which, for example, provides software-based assistance in managing a gas detection instrument or a plurality/fleet of gas detection instruments and accessories therefor (such as the Grid Fleet Manager available from Safety io of Cranberry Township, PA). Such a software-based system or grid 500 (see FIG. 2) may, for example, be in communicative connection with or implemented upon one or more computers/networks located at a facility or located remotely as part of an overall network or system 5. However, as described above, the devices, systems and methods hereof for accessory device identification and analysis may be integrated into a gas detection instrument or device 10 which is not in operative connection or integrated with such a networked system.

In a number of embodiments, software-based management system 500 is a cloud-based system in communicative connection with one or computers/networks and/or devices located at a facility. For example, in a number of embodiments, management system 500 is a remote, cloud-based system via which a particular facility may create an account to enable use of the software-based management system to assist the facility in consolidating and streamlining gas detection related activities in a single source, thereby facilitating information access, instrument/accessory compliance, risk assessment, and worker compliance. A cloud-based system may also facilitate sharing of information or data with a remote location (for example, of a manufacturer of gas detection instruments and accessories therefor) to, for example, further process data and/or provide enhanced functionality.

Management system 500, which may, for example, include a computer or a plurality of interconnected/networked computers, provides a centralized destination for managing/monitoring the facility's fleet of gas detection instruments and accessories therefor. In a number of embodiments, management system 500 is accessible, for example, via account login from facility computers, mobile devices etc. One may, for example, provide real-time notifications, instrument configuration, fleet management and relevant contextual reporting via system/grid. Management system 500 may, for example, execute a web-based application to transmit data to system 5 and received data from system 5.

FIG. 2 illustrates an embodiment of a system 5 hereof for an entity or facility including portable gas detection devices instrument(s) 10 as well as associated equipment or accessory devices therefor. In the illustrated embodiment, system 5 includes a system 80 for instrument charging and testing which may, for example, include one or more single- and/or multiple-unit chargers, calibration stands, and gas cylinder holders to operatively connect to gas cylinders. The interaction of instrument(s) 10 with system 80 to achieve assignment to and de-assignment from particular users is, for example, described in U.S. patent application Ser. No. 17/496,420. Various wired and/or wireless communication protocols as illustrated in the representative embodiment of FIG. 2 may be used in system 5 to communicate data between facility devices/systems as well as to communicate data between facility device/systems and supervisory or management system 500. Supervisory or management system 500 includes one or more processor systems programmed with one or more algorithms stored in a memory system as, for example, described herein and in U.S. patent application Ser. No. 17/496,420.

In the illustrated embodiment, portable gas detection device 10 includes an accessory device identification system in communicative connection with the control system which may, for example, include one or more communication devices which are operable to or configured to communicate data/information. Such a communication device may, for example, be integrated into or be in operative/communicative connection with communication system 48 and/or electronic circuitry 40. The communication device or devices communicate with a corresponding or cooperating communication device of gas detection accessory device(s) 200 to be used with device 10. In a number of embodiments, the proximity of the communication instrument or device 10 and the corresponding communication device 210 of a gas detection accessory device 200 may initiate communication/transmission of data/information without user intervention beyond taking some action to effect proximity. The communication device(s) of device 10 may communicate with the corresponding communication device 210 or identifier of gas detection accessory 200 via a wired (for example, through contact of one or more conductive contact elements) or a wireless manner (for example, via electromagnetic waves such as radio waves, via hall effect switches or via an optical reading methodology such as a bar code reader). As described above, in the case that an accessory device combination is used in an assembly attached to inlet 38, each accessory device 200 in the combination may be identified sequentially in a number of embodiments. For example, each accessory device 200 may include a separate RFID tag (or other proximity device) and be brought into proximity with device 10 sequentially to identify all accessory devices 200 in the combination. In other embodiments, a combination or stack of accessory items to be used in an assembly attached to inlet 38 may, for example, be provided in the form of a kit, and a single RFID tag (or other proximity device) may be associated with the combination to transmit or provide data including identification data for the combination or stack of the assembly.

Device 10 may also or alternatively monitor (via, for example, one or more sensors) one or more operational variables or parameters such a pump motor back EMF, pressure (measured, for example, via a pressure transducer), and/or pulse width modulation parameters to identify an attached accessory device 200 or accessory device combination based on stored data for such operational variables or parameters. Additionally, time and environmental conditions such as temperature and relative humidity may be measured and tracked for identification and/or analysis. Environmental conditions may, for example, affect measured operational variables or parameters such a pump motor back EMF, pressure, and/or pulse width modulation parameters. Pump motor back EMF and/or pressure may, for example, be measured at startup for changes in motor pull-in torque as a result of pressure-drop across one or more accessory devices (for example, a sampling line etc.) characteristic of the one or more accessory devices once such accessory devices are attached to device 10. Detection of one or more operational parameters for accessory device identification may be used, for example, to decrease user interactions steps in the identification process (as compared to, for example, use of a proximity-based communication system). Typically, back EMF, pressure, and/or PWM parameters would be measured once all components of an accessory device combination were attached to device 10 and matched to a stored value or range of values for identification of the accessory device combination. However, one or more such variables or parameters may also be measured upon the sequential connection of each accessory device 200. Data of operational variables or parameters such as back EMF, pressure, and/or pulse width modulation parameters may be stored in a database hereof for individual accessory devices 200 and/or combinations of accessory devices for identification thereof (individually or collectively as described above). Such data may, for example, be stored as a function (for example, as an algorithm or as a look-up table) of environmental conditions and/or other conditions that might affect the measurement thereof.

In a number of embodiments, detection of a variable or parameter such as back EMF, pressure, and/or PWM parameters may be used as a type of backup in case, for example, a user fails to use a proximity-based (or other) methodology to identify accessory devices 200 configured to be attached to device 10. For example, if an increase in back EMF and/or pressure is detected, device 10 may prompt the user to inquire if an accessory device 200 has been connected and, if not, to determine if there is an error/malfunction in the system. If an accessory device 200 has been attached to device 10, the user may be prompted to execute an identification procedure as described herein (for example, a proximity-based procedure).

Moreover, operational variables or parameters such as back EMF, pressure, and/or pulse width modulation parameters may be used to check for proper attachment and/or functioning of identified accessory devices 200 or combinations thereof. For example, a leak in the accessory device transport path or accessory device path may result in a higher back EMF or pressure than stored in memory for the identified accessory device(s) 200. A blockage or occlusion in the accessory transport line (for example, a blocked filter) may result in a lower back EMF or pressure than stored in memory for the identified accessory device(s) 200. Data for determination of proper attachment and/or functioning of an accessory device may be stored in a database hereof and may overlap partially or completely with stored data used in device accessory identification via a device operational parameter.

In the case that an accessory device 200 or combination of accessory devices 200 is not recognized by the accessory device identification system hereof because associated identification information is not present in the database, the user may be prompted or alerted by device 10. The user may further be provided with an option to enter information regarding the unidentified accessory device(s) 200 manually (for example, in the case of a sampling line, manufacturer of the sampling line, length, diameter, etc.). Data for new accessory devices 200 or combinations thereof may be uploaded to device 10 via communication from, for example, system 5 (for example, via management system 500; for example, via software updates). Likewise, data for accessory device(s) 200 or combinations thereof existing in the database may be updated via, for example, communication through system 5 as described for new accessory device(s) 200 or combinations thereof.

Examples of wireless data communication devices suitable for use herein to communication data when in proximity include, but are not limited to, radio-frequency identification (RFID) devices and near field communication (NFC) devices. In a number of representative embodiments discussed herein, the communication device of instrument 10 includes an RFID device, reader, or chip 60 which is in operative or communicative connection with electronic circuitry 40. As known in the art, RFID systems utilize electromagnetic energy/fields to wirelessly communicate with RFID tags which are associated with objects. RFID tags include a microchip or integrated circuit to store and process information. The integrated circuit further modulates and demodulates radio-frequency or RF signals. The RFID tag further includes an antenna to receive and transmit the signal over relatively short distances. Data/information of the tag is stored in a non-volatile memory. Either fixed or programmable logic is provided for processing the transmission and sensor data.

As also illustrated in the embodiment of system 5 of FIG. 2, information associated with individual user data can be substantially automatically transferred via for example a card 300 (or other object or item carried on the person of, including worn by, the user) including a communication device such as an RFID device/tag 310. In that regard, individual user data (associated with a user such as identity, entity affiliation etc.) can be transmitted to RFID device/reader 60 of device 10 upon placing card 300 in proximity to RFID reader 60. In a number of embodiments, the data transmitted from tag includes the UUID (universally unique identifier) or other unique identification information of RFID device/tag 310. The UUID or other unique identification information of RFID tag 310 is associated with the information of the individual user in the database of management system 500. In that regard, upon removal of device 10 from a bay of charger of system 80 by a user/worker, the user's information is read (for example, from card 300 when in proximity to device 10) by device 10 and device 10 is designated as assigned to the user. Device 10 may remain assigned to the user until instrument 10 is placed in one of the bays of a validated charger of system 80 and a de-assignment occurs.

In addition to transfer of information form card 300 to device 10, information can be transferred from card 300 of the user and/or another device of system 5 to a personal communication device 400 of the user as illustrated in FIG. 2. Personal communication device 400 may, for example, be a near field communication NFC/RFID enabled device as represented schematically by element 410 in FIG. 2. Furthermore, personal communication device 400 may receive data from device 10 via RFID communication from an RFID tag 60′ or other communication instrument of device 10. Personal communication device 400 (for example, a smartphone) may, for example, be used in management of RFID tag and associating data with such tags in management system 500. In that regard, an application or app can be downloaded (for example, from management system 500) via which personal communication device 400 can be used to, for example, set up/program RFID tags or other communication devices to, for example, associate personal/identity information or data of the associated user with the UUID of RFID tag 310 or other communication device of card 300. A mobile app on personal communication device 400 may, for example, be used to download a workforce roster from management system 500. Placing personal communication device 400 into proximity with card 300 can be used to achieve pairing, for example, in the cloud.

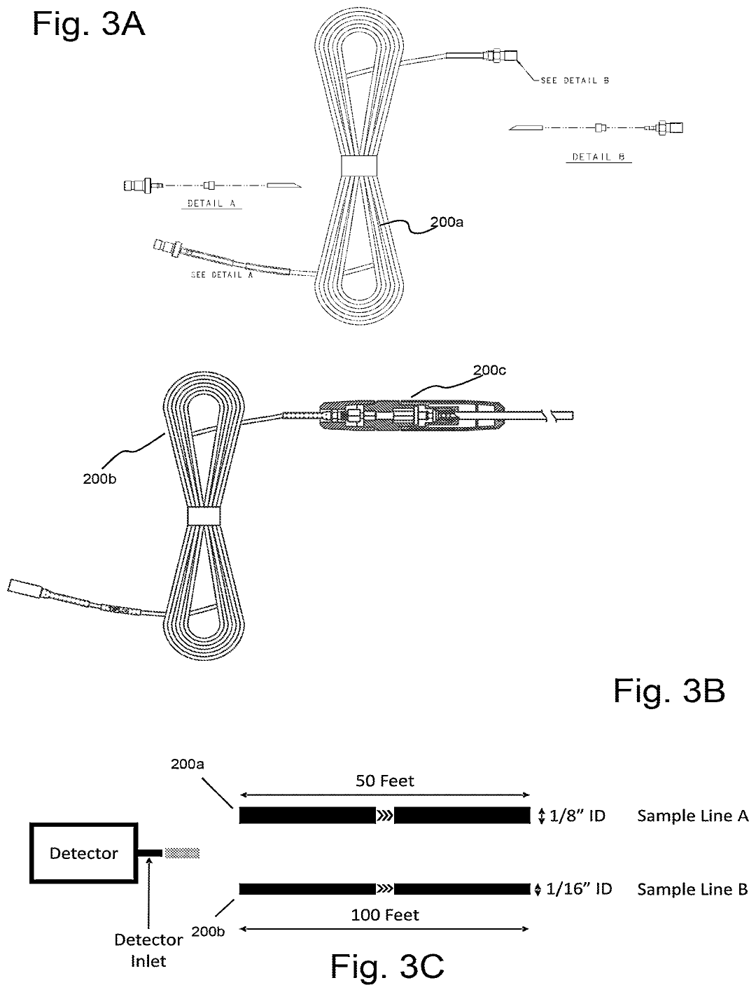

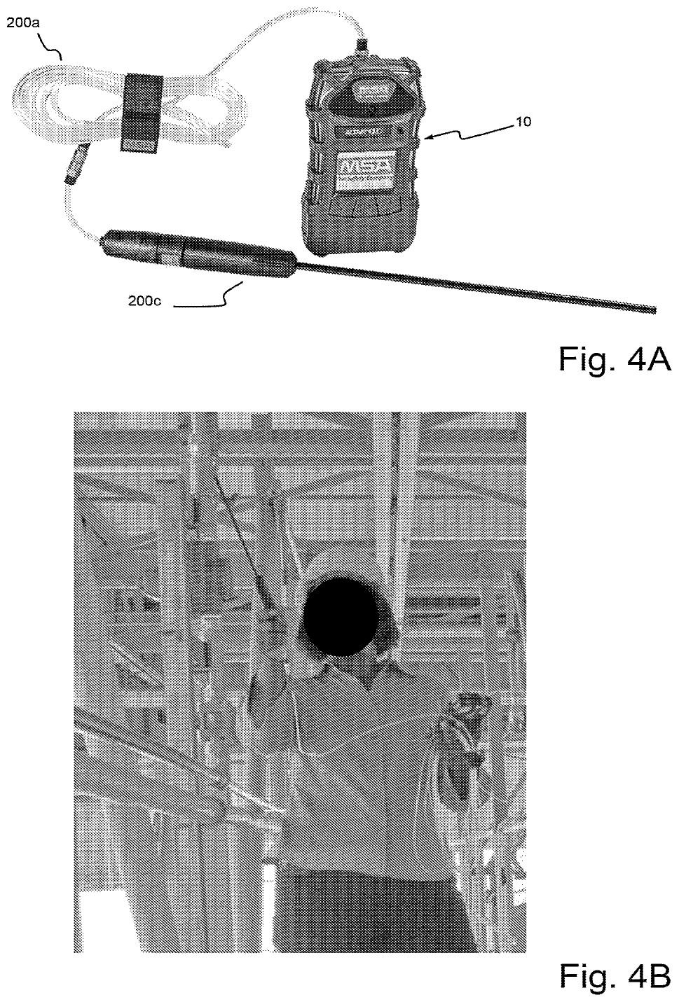

As set forth above, a number of accessory devices are desirable or required for the use of portable gas detection instruments of a gas monitoring system of an entity (for example, a company or facility). As, for example, illustrated in FIGS. 3A through 4B, portable instruments such as instrument 10 are often used in connection with various accessory devices including sampling lines and probes through which an environmental sample is pumped to the sensor via a pump system 36 in fluid connection with inlet 38 (see FIG. 1B). Tables 1 through 8 below, in connection with FIGS. 3A through 4B provide some insight into various choices of sampling lines available from a single supplier (MSA Safety Incorporated of Cranberry Township, Pennsylvania).

TABLE 1
Sampling Lines with Quick Connectors

| Product No. | Material | Length | | Diameter |
|---|---|---|---|---|
| 10040662 | Polyurethane, Coiled | 5 | ft | 1/16″ |
| 10040663 | Polyurethane | 15 | ft | |
| 10040664 | Polyurethane | 25 | ft | |
| 10040665 | Polyurethane | 10 | ft | |
| 10040666 | Polyurethane | 5 | ft | |
| 10040667 | Polyurethane, Coiled | 3 | ft | |
| 10049057 | Teflon | 25 | ft | |
| 10049058 | Teflon | 10 | ft | |

TABLE 2
Sampling lines with threaded connectors

| Product No. | Material | Length | | Diameter |
|---|---|---|---|---|
| 710217 | Polyurethane | 3 | ft | 1/16″ |
| 497332 | Polyurethane | 5 | ft | |
| 497333 | Polyurethane | 10 | ft | |
| 497334 | Polyurethane | 15 | ft | |
| 497335 | Polyurethane | 25 | ft | |
| 800972 | Teflon | 5 | ft | |
| Brass Bulkhead | | | | |
| 100745 | Teflon | 5 | ft | |
| Stainless Steel Bulkhead | | | | |
| 100181 | Polyurethane, Coiled | 3 | ft | |
| 100473 | Polyurethane | 50 | ft | |
| 100473 | Polyurethane | 25 | ft | |
| 100537 | Polyurethane | 5 | ft | |
| 100537 | Polyurethane | 10 | ft | |

TABLE 3
Conductive Sampling Lines

| Product No. | Material | Length | |
|---|---|---|---|
| 10103188 | Polyurethane, Conductive | 1.5 | m |
| 10103189 | Polyurethane, Conductive | 3 | m |
| 10103190 | Polyurethane, Conductive | 5.0 | m |

TABLE 4
Sampling Probes with Quick Connects

| Product No. | Material | Length |
|---|---|---|
| 10042621 | PEEK | 1 ft |
| 10042622 | PEEK | 3 ft |

TABLE 5
Probe and Sampling Lines with Quick Connects

| Product No. | Material | Length | Diameter |
|---|---|---|---|
| 10105210 | Polyurethane, Coiled | 5 ft | 1/16″ |
| 10105251 | Polyurethane | 5 ft | |
| 10105838 | Teflon | 10 ft | |

TABLE 6
ALTAIR Hand Probe, Clear

| Product No. | Material | Length |
|---|---|---|
| 10150844 | Hand Probe | 1 ft |
| 10153041 | Hand Probe | 1 ft |
| 10165190 | Hand Probe, w/Quick Connect | 1 ft |

TABLE 7
Conductive Sampling Probe

| Product No. | Material | Length |
|---|---|---|
| 10103191 | Sampling Probe | 1 ft |

TABLE 8
Dilution Tubes and Adapters

| Product No. | Description |
|---|---|
| 813514 | Dilution Tube |
| 100490 | Quick Connect |
| 100490 | Quick Connect |
| 10161775 | Quick Connector |
| 10053294 | Fitting, QCK Disconnect |
| 10151722 | Quick Disconnect |

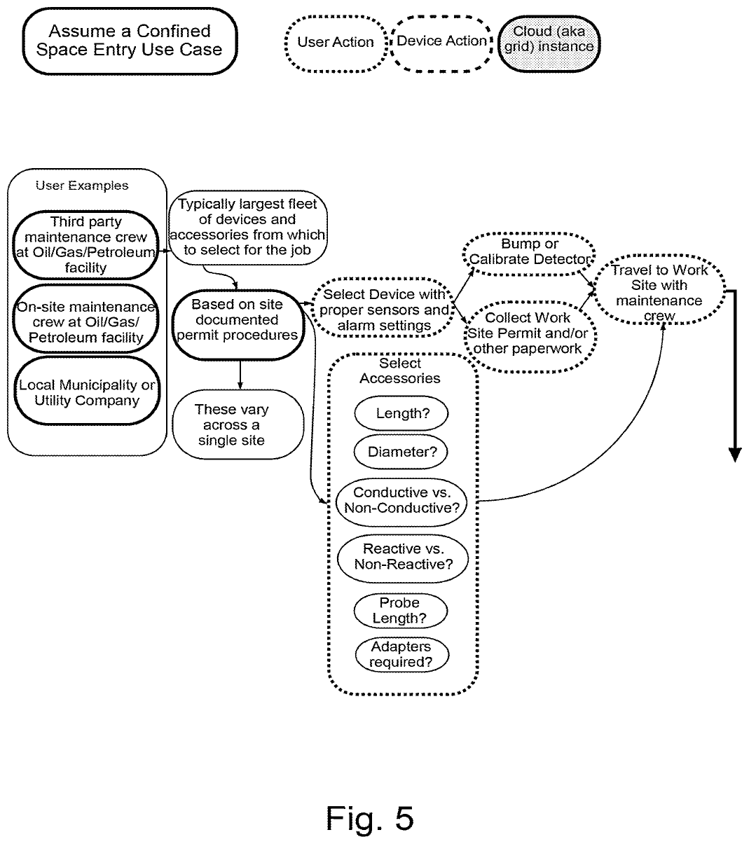

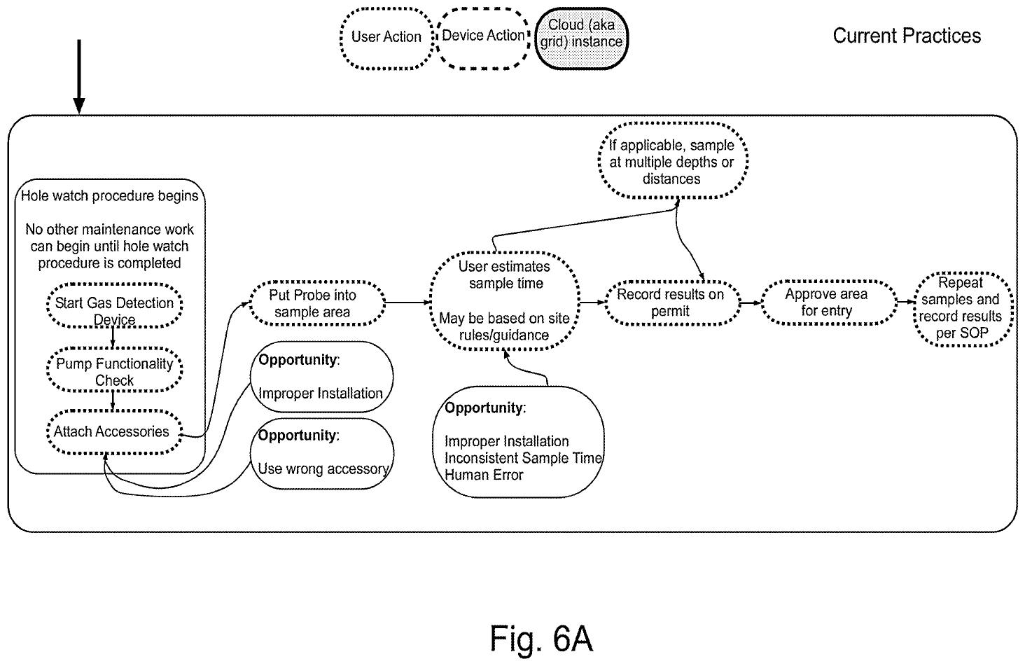

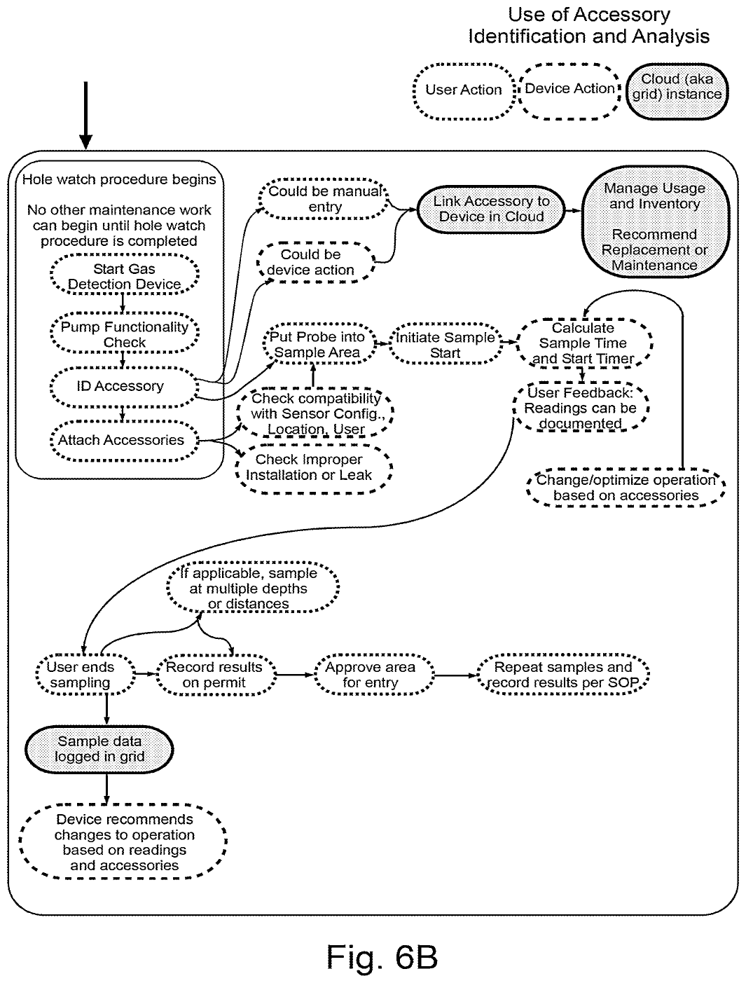

FIG. 5 illustrates a representative example of a workflow diagram for gas sensing in a confined space before entry therein using a portable gas sensing instrument or device such as device 10 and using a sampling line and a probe (and potentially one or more adapters, if required) in connection therewith. As described above, the responsibility to select a correct accessory device/accessory device combination such as a sampling line and probe for the sensor configuration of device 10 currently falls upon the user. Upon selection of an appropriate instrument or device 10 which includes proper sensors and alarm settings, selection of a sampling line, a probe and potential adapters requires choices in accessory device characteristics including, but not limited to, length, diameter, conductive versus non-conductive material, reactive versus non-reactive material, probe length, and appropriate adapter(s). The choice is complicated by the existence of numerous alternative choices for accessory device. As discussed above, Tables 1 through 8 provide representative examples of accessory device options available from a single manufacturer. As also set forth above, sampling lines and probes, as well as various adapters for use in connection therewith, are available in the market from multiple device manufacturers for a number of generations of products. As illustrated in FIG. 6A, the devices, systems and methods hereof provide a number of opportunities for improvement subsequent to accessory identification including, but not limited to, sensing and remedying of improper installation, notification of incorrect accessory device(s), reduction of the likelihood of human error in attaching (or in operating) one or more accessor devices 200 of an assembly, recommendation of or control of operation parameters of device 10 based upon identified accessory devices, etc.

FIG. 6B illustrates an embodiment of a workflow diagram using the devices, systems, and methods hereof for accessory identification and corresponding analysis/processing in system 5. After starting device 10, the user can, for example, preform a pump functionality check. In a number of embodiments, prior to attaching accessory device(s) 200, each accessory device 200 is brought into proximity with RFID device/reader 60 of instrument 10, similar to the process described above for individual user data from card 300. Data associated with one or more accessory devices 200 can be transmitted to RFID device/reader 60 of device 10 upon placing accessory device(s) 100 in proximity to RFID reader 60. In a number of embodiments, the data transmitted from RFID tag 210 includes the UUID and/or other unique identification data/information of RFID device/tag 210. The UUID or other unique identification information of RFID tag 210 is associated with and matchable with reference identification information of accessory device(s) 200 in the database of the accessory device identification system hereof. That database or database system may be a database stored in memory 44 of instrument/device 10, stored on management system 500, stored on one or more of the facility computers, or be distributed among various components of system 5. In the first instance, the identity of accessory device(s) 200 is/are determined from data stored in one or more databases. Characterization data associated with the identified accessory device(s) 200 are also stored in the database(s). Such data may, for example, include the data provided in any one of Table 1 through 8, selection criteria as set forth in FIG. 5, as well as other data for analysis such as compatibility with device 10, compatibility with other accessory device or combinations/stacks of accessory devices, parameters to determine or confirm identification or to determine proper installation, parameters for use of an accessory device or accessory device combination, device operational parameters, recommendations for optimal device operation parameters for give accessory devices or accessory device combinations, etc. Such, stored characterization data may depend on or include conditions of use (including for example, location/mode of use, environmental conditions, user etc.). Table 9 below sets forth representative data for accessory device assembly identification and representative data for characterization/analysis for two representative accessory device assemblies (wherein each such assembly includes a sampling line only, with no sampling probe or adapters). Assembly A includes a ⅛ inch diameter sampling tube of 10 feet in length. Assembly B includes a 1/16 inch diameter sampling tube of 50 feet in length. Additional characterization data may be provided as described herein and depending upon desired analysis of the use of various accessory device assemblies. Flow rate, if desired, may be measured or calculated as a function of back EMF and pressure

TABLE 9

| | | | | | | | Look-Up Table limits | |
|---|---|---|---|---|---|---|---|---|
| | | | | | | | established via | |
| | | | | | | | Characterization Testing of | |
| Accessory | | | | | | | Accessory Assembly | |
| Assembly | | Relative | | Back | | Flow | Minimum | Maximum |
| ID | Temp. | Humidity | PWM | Emf | Press. | Rate | Pressure | Pressure |
| (e.g. UUID) | (° C.) | (%) | (%) | (V) | (PSI) | (LPM) | (PSI) | (PSI) |
| Assembly A | T | RH | X_{A} | Y_{A} | P_{A} | Z_{A} | P_{A1} | P_{A2} |
| Assembly B | T | RH | X_{B} | Y_{B} | P_{B} | Z_{B} | P_{B1} | P_{B2} |

Automated or partially automated identification of an accessory device assembly including one or more accessories 200 and analysis/matching of such accessory devices 200 and combinations or stacks thereof may be used to significantly reduce the burden upon the user in selecting an accessory device 200 or combination/stacks of accessory device 200 for use with device 10. With little or no input from the user, the device, systems, and methods hereof can identify accessory device(s) 200 to be attached to device 10 and analyze/compare the information associated with such accessories 200 with, for example, the sensor configuration of device 10. In the illustrated embodiment, device 10 checks the compatibility of identified accessory device(s) 10 with the sensor configuration of instrument or device 10, the compatibility of accessory devices 200 with each other, compatibility with the location, etc. Location can, for example, be determined from GPS data, localization systems at a facility, and/or other methodologies as known in the localization arts. An alarm and/or flag to alert the user of unsafe or improper use cases can be provided to a user via, for example, device 10 and/or another component of system 5. Moreover, a determination can be made (and an alert provided) if a particular user may not be adequately qualified/trained for a particular use associated with, for example, an identified accessory device 200/accessory device combination and/or a particular location of use. Device 10, either alone, or with analysis/processing from one or more other components of system 5, can further provide the user with increased confidence of a proper setup via confirmation cues and/or instructions to assist in creating a proper or optimal set up.

A lock setting may be provided in the software stored in memory system 44 of device 10 (and or elsewhere on an associated network or grid) in a number of embodiments. If such a lock setting is enabled, device 10 may be setup to not enter a normal operating mode if a triggering state is detected such as if a noncompliant accessory device or accessory device combination is placed in connection with device 10, if a user assigned to the device is not adequately qualified/trained as described above, and/or if improper attachment or function of accessory device(s) is detected based upon pre-established and stored criteria. In a number of embodiments, if the lock setting is enabled and a triggering state is detected (for example, a noncompliant accessory device 200 or accessory device combination is identified), device 10 will go into locked state. Alternatively or additionally an alarm or warning can be provided to the user. In a number of embodiments, device 10 may return to an unlocked or operational state once a triggering state is eliminated or overridden.

Identification of accessory devices 200 may provide additional benefits in operating efficiency and safety in devices 20. For example, a prompt or warning may be provided to change one or more accessory devices 200 if one or more gases is detected by a sensor 30 of device 10 or another sensor/device of system 5 in a relevant location (for example, a sensor of a fixed device or another portable device) that is/are chemically incompatible with an identified accessory device 100 or incompatible with a relevant safety regulation or protocol (which may change depending upon location).

Identification of accessory devices 200 may be used to control or alter the function device 10 (for example, the manner in which menus are presented). Identification of accessory devices 200, and determination of compatibility and proper installation thereof by device 10 may, for example, cause device 10 to initiate or enter the sampling mode of device 10 or to prompt the user to enter the sampling mode. Entering a sampling mode in the case of a number of currently available devices 10 may, for example, require navigation through several menus/submenus. Identification of accessory device(s) 200 to be attached to a device 10 (and, optionally, determination of proper attachment thereof in certain embodiments), may, for example, automatically display a menu option for the user to enter the sampling mode, thereby simplifying and streamlining the operation of device 10.

Identification of accessory device(s) 200 may be used to achieve efficiency gains for sampling via stored knowledge or characterization data based upon, for example, line length, diameter, material properties, probe, etc. as described above. Under current practices, for example, a user must determine how much time to wait before a reading should be taken via device 10. In that regard, sufficient time must pass before a sample pumped from an environment to be tested reaches sensors 30. A rule of thumb such as a defined period of time of delay (for example, 2 or 3 seconds) for each foot or meter of sampling line length may be used. In that regard, the amount of time a sample from an environment being tested reaches sensors 30 of device 10 increases with increasing length of a sampling line accessory such as sampling lines 200a and 200b illustrates in FIGS. 3A and 3B. Other variables such as sampling line diameter also affect transit time of samples from a sampled environment. Likewise, different probes (such as probe 200c of FIG. 3B) may result in different sample flow characteristics. Upon identification of accessory devices 200, device 10 may, for example, calculate the sample time based on the saved characteristics of the identified accessory devices 200. Data of environmental conditions affecting flow such as humidity, temperature, pressure, and/or altitude from various sensors that may be incorporated in device 10 or data transmitted to device 10 from another device of system 5 may also be used in determining sample time. Thus, in addition to improvement or optimization of accessory setup, the operation of device 10 may be improved or optimized on the basis of identification of accessory device(s) 200.

The user typically ends sampling after following standard operating procedures (SOP) for a particular location/site and the results are recorded. In the case of a confined entry site, sampling may, for example, occur at multiple depths or distances. Sampling may also be repeated. Data recorded may, for example, include start time and levels measured at the start of monitoring, level readings at east depth/distance, continuous air monitoring at a given time increment and time of entry into the confined space after approval for entry.

An additional benefit of accessory device identification hereof is that accessory devices may be readily asset managed via software-based management system or grid 500. Sample data may, for example, be automatically uploaded to management system or grid 500. Management system or grid 500, subsequent to identification accessory devices 200 being associated with or attached to a device 10, enables tracking of which accessory device(s) 200 are (or have been) associated with each instrument (as well as used by which user(s)) for particular time periods, how many accessories have been used, the frequency of use of each accessory and/or accessory combination etc. Accessory device management provided by management system or grid 500 can thereby assist a facility in understanding and track usage and needs. Further, misuse may be identified if an incorrect accessory device is being used in a specific sampling location. For example, specific locations can be designated as requiring specific accessory setups and GPS or other device location during sampling may trigger an alarm if the incorrect setup is being used as described above. Needs for further training for all or one or more particular user may be identified.

Tracking the use of device 10 and accessory devices 100 used in connection thereof, as well as sample data (includes exposure to various gases) may, for example, be used in diagnosing problems with devices 10, scheduling calibration or maintenance procedures, or in estimating device lifetime. Such data may further be used in optimization algorithms for improving methodologies, device 10, accessory devices 200, and or combinations thereof.

The foregoing description and accompanying drawings set forth a number of representative embodiments at the present time. Various modifications, additions and alternative designs will, of course, become apparent to those skilled in the art in light of the foregoing teachings without departing from the scope hereof, which is indicated by the following claims rather than by the foregoing description. All changes and variations that fall within the meaning and range of equivalency of the claims are to be embraced within their scope.

What is claimed is:

1. A system, comprising:
a gas detection device comprising a housing, a control system comprising a processor system and a memory system in communicative connection with the processor system, one or more gas sensors within the housing in communicative connection with the control system, wherein each of the one or more gas sensors is independently responsive to a gas analyte, an inlet via which gas to be sampled from an environment enters the housing to come into contact with the one or more gas sensors, and a pump system in fluid connection with the inlet and in fluid connection with the one or more gas sensors, and
an accessory device identification system in communicative connection with the control system of the gas detection device, wherein the accessory device identification system is configured to receive data including identification data for at least one of (i) each of one or more associated accessory devices or (ii) an associated combination of accessory devices configured to be placed in fluid connection with the inlet of the gas detection device, the accessory device identification system further being configured to identify at least one of (i) each of the one or more associated accessory devices or (ii) the associated combination of accessory devices from the identification data received.

2. The system of claim 1 wherein the accessory device identification system further comprises a database having stored therein data for at least one of a plurality of accessory devices or a plurality of combinations of accessory devices including reference identification data for at least one of each of the plurality of accessory devices or each of the plurality of combinations of accessory devices, the accessory device identification system being configured to compare the identification data received with the reference identification data to identify (i) each of the one or more associated accessory devices or (ii) the associated combination of accessory devices.

3. The system of claim 2 wherein the database further has stored therein characterization data for at least one of each of the plurality of accessory devices or each of the plurality of combinations of accessory devices.

4. The system of claim 2 wherein the database is stored in the memory system of the control system.

5. The system of claim 2 wherein each the one or more associated accessory devices or the associated combination of accessory devices includes an identifier in operative connection therewith, the identifier being configured to provide the data including the identification data to the accessory device identification system.

6. The system of claim 2 wherein the gas detection device further comprises a sensing system in operative connection with the control system, the sensing system being configured to measure a property of the pump system which varies upon attachment of the one or more associated accessory devices or the associated combination of accessory devices to the inlet, data of the property of the pump system being used in at least one of: identifying the one or more associated accessory devices or the associated combination of accessory devices, wherein the reference identification data comprises the data of the property of the pump system, determining a state of attachment of the one or more associated accessory devices or the associated combination of accessory devices, or determining a state of functionality of the one or more associated accessory devices or the associated combination of accessory devices.

7. The system of claim 4 wherein each of the one or more associated accessory devices or the associated combination of accessory devices includes an identifier in operative connection therewith, the identifier being configured to provide the data including the identification data to the accessory device identification system via a communication device of the accessory device identification system.

8. The system of claim 4 wherein the gas detection device further comprises a sensing system in operative connection with the control system and configured to measure a property of the pump system which varies upon attachment of the one or more associated accessory devices or the associated combination of accessory devices to the inlet, the data of the property of the pump system being used in at least one of: identifying the one or more associated accessory devices or the associated combination of accessory devices, wherein the reference identification data comprises the data of the property of the pump system, determining a state of attachment of the one or more associated accessory devices or the associated combination of accessory devices, or determining a state of functionality of the one or more associated accessory devices or the associated combination of accessory devices.

9. The system of claim 7 wherein the identifier comprises an RFID tag and the communication device of the accessory device identification system comprises an RFID reader.

10. The system of claim 7 wherein the gas detection device further comprises a sensing system in operative connection with the accessory device identification system and configured to measure a property of the pump system which varies upon attachment of the one or more associated accessory devices or the associated combination of accessory devices to the inlet, the data of the property of the pump system being used in at least one of: identifying the one or more associated accessory devices or the associated combination of accessory devices, wherein the reference identification data comprises the data of the property of the pump system, determining a state of attachment of the one or more associated accessory devices or the associated combination of accessory devices, or determining a state of functionality of the one or more associated accessory devices or the associated combination of accessory devices.

11. The system of claim 7 wherein the memory system further comprises software stored thereon and executable by the processor system to compare the identification data received with the reference identification data to identify the one or more associated accessory devices or the associated combination of accessory devices.

12. The system of claim 11 wherein the database further has stored therein characterization data for at least one of the plurality of accessory devices or the plurality of combinations of accessory devices, and the memory system further comprises analysis software stored thereon which is executable by the processor system to analyze the one or more associated accessory devices or the associated combination of accessory devices.

13. The system of claim 12 wherein the analysis software is configured to determine at least one of compatibility of the one or more associated accessory devices or the associated combination of accessory devices with the gas detection device, the compatibility of one associated accessory devices with another associated accessory devices, compatibility of the one or more associated accessory devices or the associated combination of accessory devices with a location, compatibility of the one or more associated accessory devices or the associated combination of accessory devices with a user, compatibility of the one or more associated accessory devices or the associated combination of accessory devices with an environment to be tested, a state of connection of the one or more associated accessory devices or the associated combination of accessory devices to the inlet, a parameter of operation of the gas detection device for use with the one or more associated accessory devices or the associated combination of accessory devices, a recommendation for changing at least one associated accessory device, or a change in operation of the portable gas detection device.

14. The system of claim 3 further comprising a management system comprising a management processor system, a management communication system configured to be in communicative connection with a gas monitoring system of an entity which comprises the gas detection device, and a management memory system in operative connection with the management processor system, the management memory system having stored therein an algorithm executable by the management processor system to at least one of track usage of or manage the gas detection device and at least one of the one or more associated accessory devices or the associated combination of accessory devices.

15. A method for use with a gas detection device including a housing, a control system including a processor system and a memory system in communicative connection with the processor system, one or more gas sensors within the housing in communicative connection with the control system, wherein each of the one or more gas sensors is independently responsive to a gas analyte, an inlet via which gas to be sampled from an environment enters the housing to come into contact with the one or more gas sensors, and a pump system in fluid connection with the inlet and in fluid connection with the one or more gas sensors, the method, comprising:
providing an accessory device identification system in communicative connection with the control system of the gas detection device, the accessory device identification system being configured to receive data including identification data for at least one of (i) each of one or more associated accessory devices or (ii) an associated combination of accessory devices configured to be placed in fluid connection with the inlet of the gas detection device, and
identifying at least one of (i) each of the one or more associated accessory devices or (ii) the associated combination of accessory devices from the identification data received.

16. The method of claim 15 wherein the accessory device identification system comprises a database having stored therein data for at least one of a plurality of accessory devices or a plurality of combinations of accessory devices including reference identification data for at least one of each of the plurality of accessory devices or each of the plurality of combinations of accessory devices, the accessory device identification system being configured to compare the identification data received with the reference identification data to identify (i) each of the one or more associated accessory devices or (ii) the associated combination of accessory devices.

17. The method of claim 16 wherein the database further has stored therein characterization data for at least one of each of the plurality of accessory devices or each of the plurality of combinations of accessory devices.

18. The method of claim 16 wherein the database is stored in the memory system of the control system.

19. The method of claim 16 wherein each the one or more associated accessory devices or the associated combination of accessory devices includes an identifier in operative connection therewith, the identifier being configured to provide the data including the accessory device identification data to the identification system.

20. The method of claim 16 wherein the gas detection device further comprises a sensing system in operative connection with the control system and configured to measure a property of the pump system which varies upon attachment of the one or more associated accessory devices or the associated combination of accessory devices to the inlet, the method further comprising using the date of the property of the pump system to at least one of: (i) identify the one or more associated accessory devices or the associated combination of accessory devices, (ii) determine a state of attachment thereof, or (iii) determine a state of functionality thereof.
